(19) United States
(12) Patent Application Publication
(10) Pub. No.: US 2006/0105941 A1
(43) Pub. Date: May 18, 2006

(54) Mixed antibiotic codrugs

(75) Inventors: Rhett M. Schiffman, Laguna Beach, CA (US); Richard Graham, Irvine, CA (US); David Rupp, San Pedro, CA (US); Brent A. Johnson, Ladera Ranch, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN, INC., Irvine, CA (US)

(21) Appl. No.: 10/988,384

(22) Filed: Nov. 12, 2004

(51) Int. Cl. A61K 38/14; A61K 31/704; A61K 31/496; A61K 31/65; A61K 31/43; A61K 31/545

(52) U.S. Cl. 514/8; 514/35; 514/152; 530/322; 536/16.8; 544/363; 514/200; 514/192; 540/222; 514/253.08

(57) ABSTRACT

Novel compounds which degrade in vivo into two or more different active antibiotics are disclosed herein. Methods, compositions, and medicaments related thereto are also disclosed.

### FIELD OF THE INVENTION

This invention relates to pharmaceutical compounds. In particular, this invention relates to antibiotic compounds.

### BACKGROUND OF THE INVENTION

### DESCRIPTION OF RELATED ART

Due to bacterial resistance to antibiotics, there is a constant need for new antibiotic compounds. Recently, Huberschwerlen, et. al. published findings for a new class of hybrid antibiotics having the structure shown below (Hubschwerlen et. al. Biorganic & Medicinal Chemistry Letters 2003, 13, 4229-4233; Hubschwerlen et. al. Biorganic & Medicinal Chemistry Letters 2003, 11, 2313-2319; WO03032962; WO03031441; and WO03031443). The authors demonstrated that these compounds are active with a wide variety of spacers comprising 4-6 membered rings. The portion of the molecule to the left of the spacer corresponds to an oxazolidinone antibiotic, and the portion of the molecule to the right of the spacer corresponds to a fluoroquinoline antibiotic.

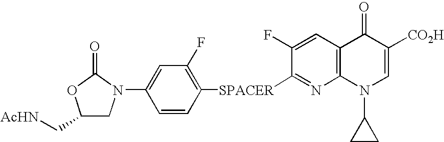

The spacers tested comprised four, five, or six membered rings having an oxygen or nitrogen that was directly attached to the oxaxolidinone portion and a nitrogen which attached directly to the fluoroquinoline portion. The fluoroquinoline was generally attached directly to the ring, i.e. the nitrogen atom to which it was attached was part of the ring. In one case, the oxazolidinone was directly attached to the ring, but most of the molecules tested had the ozalolidinone attached to a nitrogen or oxygen that was attached as a substituent to the ring, or the nitrogen or oxygen was connected to the ring by —CH₂— or —(CH₂)₂—. The groups shown below are typical examples, where the dashed lines indicate the bonds attaching to the two antibiotics

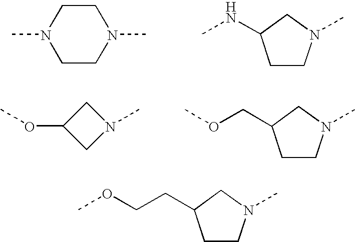

Most of the compounds reported had oxazolidinone activity and fluoroquinolone activity.

Compounds which degrade in vivo into two or more active drugs have been called mutual prodrugs, drug conjugates, and codrugs. A review of the earliest mutual prodrugs prepared and tested was published a decade ago by Gurpartap and Sharma (Indian Journal of Pharmaceutical Sciences, 1994, 63(3), pp. 69-79).

U.S. Pat. No. 6,051,576, which issued on Apr. 18, 2000, claims “A sustained release, and substantially inactive codrug, comprising at least two drugs ionically or covalently linked to one another wherein each active drug is regenerated upon bond cleavage.” The patent further states:
A codrug of the invention may consist of one or more pharmacologically active compounds in the following classes of agents; anesthetics and pain killing agents such as lidocaine and related compounds and benzodiazepain and related compounds; anticancer agents such as 5-fluorouracil, adriamycin and related compounds; anti-inflammatory agents such as 6-mannose phosphate; anti-fungal agents such as fluconazole and related compounds; antiviral compounds such as trisodium phophomonoformate, trifluorothymidine, acyclovir, ganciclovir, dideoxyinosine (ddI), dideoxycytidine (ddC); cell transport/mobility impeding agents such as colchicine, vincristine, cytochalsian B and related compounds; anti-glaucoma drugs such as carbonic anhydrase inhibitors, beta blockers, miotics, cholinesterase inhibitors, and sympathomimetics; immunological response modifiers such as muramyl dipeptide and related compounds; cytokines and peptides/proteins such as cyclosporin, insulin, growth factor or growth hormones and steroids. Non steroidal anti-inflammatory agents include, for example, flurbiprofen and indomethacin.

U.S. patent application Ser. No. 6,051,576, discloses codrugs wherein the two drugs linked are
selected from antidepressant compounds, analgesic compounds, anti-inflammatory steroidal compounds (corticosteroids), non-steroidal antiinflammatory compounds (NSAIDs), antibiotic compounds, anti-fungal compounds, antiviral compounds, antiproliferative compounds, antiglaucoma compounds, immunomodulatory compounds, cell transport/mobility impeding agents, cytokines and peptides/proteins, skin-treating compounds, sunscreens, skin protectants, antimetabolite compounds, antipsoriatic compounds, keratolytic compounds, anxiolytic compounds, and antipsychotic compounds.

Macky and coworkers (J. Med. Chem., 2002, 45, 1122-1127) described a mitomycin C and triamcinolone acetonide conjugate which used glutaric acid as a linker.

### BRIEF DESCRIPTION OF THE INVENTION

A compound comprising two antibiotics belonging to distinct classes, which are connected via two covalent bonds to a linker such that said compound degrades in vivo to yield the two antibiotics, wherein each bond is an amide bond or an ester bond is disclosed herein.

A compound which is an active antibiotic, which degrades in vivo into two or more smaller active antibiotics belonging to distinct classes, is also disclosed herein.

A compound comprising a linker having two bonds, wherein said bonds are asymmetrically degraded in vivo to release the two antibiotics belonging to distinct classes is also disclosed herein.

A compound comprising 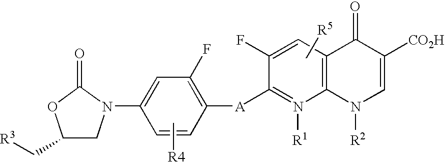
or a pharmaceutically acceptable salt or a prodrug thereof;

wherein A is a linking group comprising an ester or an amide bond X is C or N;

R¹ and R² are independently H, C₁₋₆ alkyl, or C₁₋₆ alkoxy, wherein R¹ and R² may be bonded such that a ring is formed;

R³ is H, C₁₋₆ alkyl, C₁₋₆ acyl, guanidinyl, C₂₋₆ alkylguanidinyl, or C₁₋₆ NH-acyl; and

R⁴ and R⁵ are fluoro, chloro, bromo, nitro, CN, CO₂H, OH, C₁₋₆ alkyl, or C₁₋₆ alkoxy, is also disclosed herein.

A method comprising linking two different antibiotics such that a mixture of isomers is formed, wherein one or both antibiotics have more than one linkable group,

a. separating said mixture into two or more fractions,

b. testing the antibiotic activity of said fractions, and

c. repeating steps b and c on the more active fractions;

wherein said method is useful for isolating or identifying a compound which is an active antibiotic, is also disclosed herein.

Methods, compositions, and medicaments related thereto are also disclosed.

### BRIEF DESCRIPTION OF THE DRAWING FIGURES

FIG. 1 illustrates a method of isolating or identifying a compound which is an active antibiotic made by linking two different antibiotics wherein one or both antibiotics have more than one linkable group.

FIGS. 2-8 illustrate possible methods of preparing compounds disclosed herein.

### DETAILED DESCRIPTION OF THE INVENTION

The two antibiotics of the compounds disclosed herein are connected via two covalent bonds to a linker such that said compound degrades in vivo to yield the two antibiotics, wherein each bond is an amide bond or an ester bond. In other words, the linker has one amide bond connecting to one antibiotic and one ester bond connecting to the other antibiotic. Alternatively, the linker is bonded to both antibiotics via ester bonds, or the linker is bonded to both antibiotics via amide bonds. The terms “ester bond” and “amide bond” have the meanings understood in the art, i.e. they are the bonds formed by the dehydration of the appropriate acid and alcohol, or the appropriate acid and amine. The determination of whether a bond is an ester bond or an amide bond is strictly a mental exercise, and is independent of the way the bond is actually formed in the preparation of the molecule, or whether or not formation of the bond by dehydration is synthetically feasible. Additionally, for the purposes of compounds disclosed herein, a bond between an amide nitrogen and another carbonyl group is also considered an amide bond. In other words, a nitrogen atom may have two amide bonds to different geminal carbonyl carbons.

Degradation of the ester or amide bonds generally, but not necessarily, yields the corresponding acid and alcohol or amine by hydrolysis or a related reaction. A compound which degrades in vivo to yield the two antibiotics produces both the antibiotics belonging to distinct classes at some point in the metabolic process of the claimed compound. In many cases, cleavage of the first amide or ester bond will release one active antibiotic, and cleavage of the second amide or ester bond will release the second antibiotic. However, cleavage of one of these bonds may yield a prodrug of one of the antibiotics, which forms the active antibiotic upon further metabolism. Alternatively, the linker may not necessarily first cleave at the ester or amide bond, but may comprise other biologically labile bonds which cleave before either or both of the ester or amide bonds.

The linker may be referred to according to its parent compound, i.e. the compound which is converted into the linker via the functional groups incorporated into the amide or ester. For example, in the example below, where A—CO₂H and B—NH₂ are antibiotics, the linker is referred to as lactic acid (CH₃CHOHCO₂H). 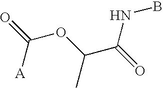
Again, the identity of the linker is strictly a mental determination, and is not dependent upon whether the compound is formed by making the designated bonds between the linker and the two antibiotics. Additionally, the linker is not dependent upon whether it is formed during hydrolysis, as it is conceivable that other compounds may be formed in vivo, and that that the linker may have additional labile bonds which are degraded before the bonds to the antibiotics degrade.

The linker may be an amino acid, where amine forms an amide bond, and the carboxylic acid forms an ester bond. Such is likely to be the case with the amino acids such as glycine, alanine, valine, leucine, methionine, proline, and phenylalanine, which contain no side chains which may be incorporated into an ester or amide bond. Alternatively, amino acids such as aspartic acid and glutamic acid have an additional carboxylic acid which may be incorporated into a carboxylic acid ester or amide. Other amino acids such as tryptophan, lysine, arginine, and histidine, contain additional amine groups which may be incorporated into amide bonds. Other amino acids such as serine, threonine, and tyrosine, contain hydroxy groups which may be incorporated into ester bonds.

The linker may also be a biological alcohol and/or acid. A number of biological compounds have two or more hydroxy groups such as sugars and other carbohydrates, glycerine, and the like. Other biological compounds have two or more carboxylic acid functional groups such as succinic acid, fumaric acid, oxaloacetic acid, ketoglutaric acid, and the like. Additionally, many biological compounds contain both carboxylic acid and hydroxy groups such as lactic acid, citric acid, isocitric acid, malaic acid, sugar acids, and the like.

However, the linker need not be of biological origin, compounds such as ethylene glycol, or oligomers or polymers thereof are also useful.

Any of the above may also be combined with one another via ester, amide, ether, or similar bonds to form a linker. A polyethylene glycol acid (PEG acid) such as 3-PEG-butyric acid, is an example of such a linker.

If the linker has two bonds which are asymmetrically degraded in vivo, one bond is broken, hydrolyzed, cleaved, or otherwise destroyed significantly more rapidly than the second, such that a prodrug of the second antiobiotic is formed. This prodrug comprises the second antibiotic bonded to the remaining part of the linker. While not intending to limit the scope of the invention in any way, asymmetric in vivo degradation confers greater flexibility to the combination in terms of control of drug release and drug delivery. While not intending to limit the scope of the invention in any way, compounds which have both an amide bond and an ester bond are will often be degraded asymmetrically in vivo due to the different chemical properties of the two functional groups.

The antibiotics may be any art recognized antibiotics which have functional groups that can be obtained by degradation of an amide or an ester bond in vivo. Such functional groups may include, but are not limited to, hydrolysis products such as carboxylic acid, hydroxy, and amino. However, it is possible that other mechanisms may operate in vivo to convert amides or esters to other functional groups, and antibiotics comprising these functional groups may also be used.

The compounds disclosed herein comprise two antibiotics belonging to distinct classes.

One class of antibiotics is the Fluoroquinolones, which includes, but is not limited to the following: levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, and sitafloxacin.

Another class of antibiotics is the Oxazolidinones, which includes, but is not limited to, linezolid, AZD2563, eperezolid, DA-7867 (Dong-A Pharmaceutical Co., Yongin, Korea), and the like.

Another class of antibiotics is Carbapenems including, which includes, but is not limited to, meropenem, ertapenem, imipenem, ME1036, and the like.

Another class of antibiotics is Cephalosporins, which included, but is not limited to the following: loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, and cephalothin.

Another class of antibiotics is Glycopeptides, which includes, but is not limited to, oritavancin, dalbavancin, vancomycin, telavancin, teicoplanin, and related drugs.

Another class of antibiotics is Macrolides, which includes, but is not limited to, erythromycin, clarithromycin, azithromycin, dirithromycin, and the like.

Another class of antibiotics is Tetracyclines, which includes, but is not limited to, minocycline, doxycycline, tetracycline, and the like.

Another class of antibiotics is Aminogycosides which includes, but is not limited to, tobramycin, streptomycin, gentamicin, kanamycin, amikacin, netilmicin, and the like.

Another class of antibiotics is Penicillins, which includes, but is not limited to, penicillin g, ticarcillin, methicillin, phenthicillin, cloxacillin, dicloxacillin, nafcillin, oxacillin, and the like.

Another class of antibiotics is Aminocyclitols, which includes, but is not limited to, spectinomycin, trospectinomycin, and the like.

Another class of antibiotics is Ansamycins, which includes, but is not limited to Rifampin and related drugs.

Another class of antibiotics is Chloramphenicol and related drugs.

Another class of antibiotics is Nubiotics, which are protonated nucleic acid-based drugs shown to have potent in vitro antibacterial activities against a number of gram-positive and gram-negative bacteria.

Another class of antibiotics is Quinolones, which includes, but is not limited to, nalidixic acid, cinoxacin, and the like.

Another class of antibiotics is Folate Antagonists, which includes, but is not limited to, trimethoprim, sulfonamide, sulfamethoxazole, and the like.

Another class of antibiotics is Fosfomycin and related drugs.

Another class of antibiotics is Glycylcyclines, which includes, but is not limited to, tigecycline and related drugs.

Another class of antibiotics is Glycolipodepsipeptides, which includes, but is not limited to, ramoplanin and related drugs.

Another class of antibiotics is Mannopeptimycins.

Another class of antibiotics is Lincosamide, which includes, but is not limited to, clindamycin and related drugs.

Another class of antibiotics is 5-Nitroimidazole, which includes, but is not limited to, metronidazole and related drugs.

Another class of antibiotics is Peptide Deformylase Inhibitors, which includes, but is not limited to, actinonin, BB-3497, and related drugs.

Another class of antibiotics is Streptogramins, which includes, but is not limited to, dalfopristin, quinupristin, and related drugs.

Another class of antibiotics is Lipopeptides, which includes, but is not limited to, daptomycin and related drugs.

Another class of antibiotics is Ketolides, which includes, but is not limited to, telithromycin and related drugs.

Another class of antibiotics is Heteroaromatic polycyclic (HARP) antibiotics, a class of small DNA-binding antibiotics, which includes, but is not limited to, GSQ1530 and related drugs.

Another class of antibiotics is Monobactams, which includes, but is not limited to, aztreonam and related drugs.

Another class of antibiotics is Bacitracin and related drugs.

Another class of antibiotics is Polymyxin and related drugs.

Another class of antibiotics is Phenyl-thiazolylurea-sulfonamides, a novel class of potent inhibitors of Phenylalanyl (Phe)-tRNA synthetase (Phe-RS).

Another class of antibiotics is Carboxypenicillins, which includes, but is not limited to, tricarcillin, carbenicillin, and related drugs.

Another class of antibiotics is Ureidopencillins, which includes, but is not limited to, azlocillin, mezlocillin, piperacillin, and related drugs.

Another class of antibiotics is Aminopenicillins, which includes, but is not limited to, bacampicillin, ampicillin, amoxicillin, and related drugs.

Another class of antibiotics is Beta-lactams, which includes, but is not limited to, faropenem and related drugs.

Another class of antibiotics is Nitrofurantoin, which includes, but is not limited to, nitrofurantoin and related drugs.

Another class of antibiotics is Anti-mycobacteria drugs.

Another class of antibiotics is Ethambutol and related drugs.

Another class of antibiotics is Isoniazid and related drugs.

In one embodiment, the antibiotics comprise a fluoroquinone and a tetracycline, such as in Compound 1 below.

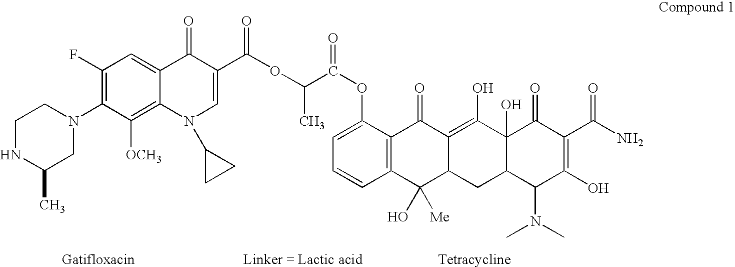

In another compound, the antibiotics comprise a carbapenem and an aminoglycoside, such as in Compound 2 below.

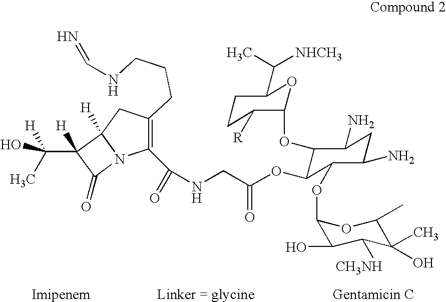

In another compound, the antibiotics comprise an oxazolidinone and an aminoglycoside, such as in Compound 3 below.

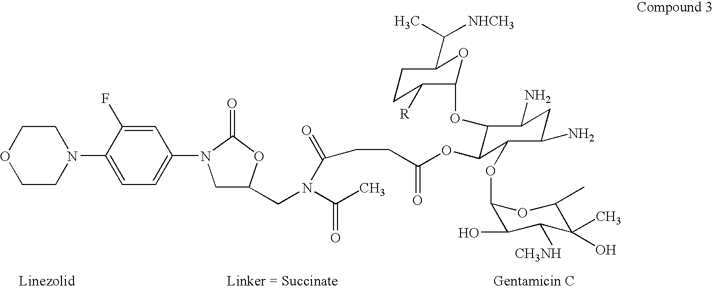

In another compound, the antibiotics comprise a cephalosporin and a fluoroquinolone, such as in Compound 4 below.

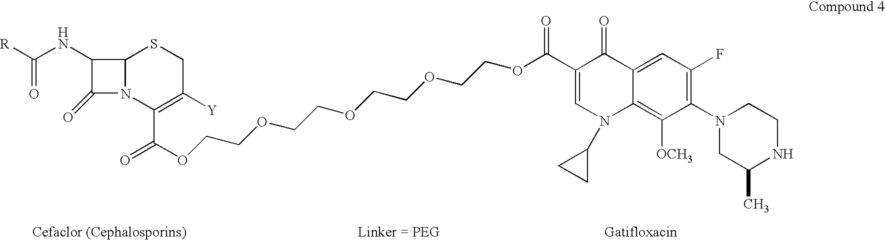

In another compound, the antibiotics comprise vancomycin and a fluoroquinolone, such as in Compound 5 below.

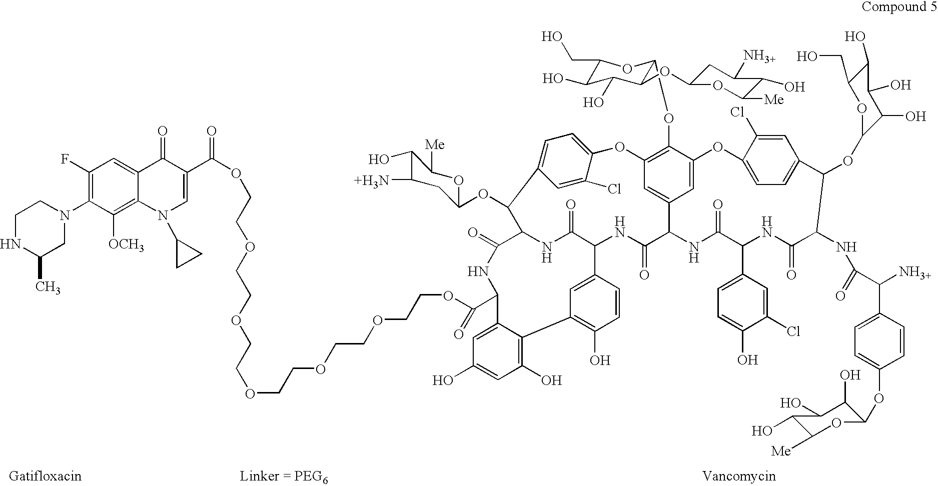

In another compound, the antibiotics comprise a macrolide and a penicillin, such as in Compound 6 below.

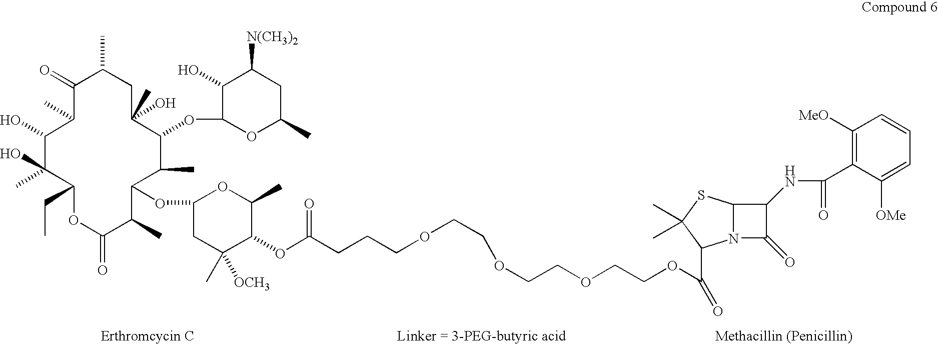

While not intending to be bound in any way by theory, it is believed many of these compounds are active antibiotics which also degrade in vivo into two or more smaller active antibiotics belonging to distinct classes. In other words, the compounds themselves act as antibiotics, and over time the antibiotics degrade in vivo to two or more different antibiotics, which are not the parent compound.

Additionally, other compounds which may not have two bonds to a linker, but merely one labile ester bond or amide bond, are contemplated herein as being active antibiotics which degrade in vivo into two or more smaller active antibiotics. While not intending to limit the scope of the invention in any way, compound 7 which is described hereafter is believed to be such a compound.

While not intending to be bound in any way by theory, it is believed that in many cases the pharmacophore of an antibiotic will not comprise the entire molecule. Thus, while not intending to be bound by theory, it is believed that some of the linkable functional groups and the surrounding atoms on an antibiotic may be bonded to a linker and another antibiotic while still retaining antibiotic activity. This belief is supported by the fact that many classes of antibiotics have a broad variety of active structures. Since this probability is substantial, a significant number of molecules prepared by linking to antibiotics as described herein will be active antibiotics. This belief is also supported by the aforementioned Hubschwerlen, et. al. work.

Compounds which are active antibiotics before degradation in vivo may be prepared, identified, or isolated by the following method. Linking of any pair of antibiotics wherein one or both of the antibiotics have multiple linkable functional groups is carried out without isolation of isomers. The mixture of isomers is then tested for antibiotic activity. If any antibiotic activity is detected, the mixture is then separated into two different fractions according to any method used in the art such as chromatography, distillation, or the like. The fractions are then tested for antibiotic activity, the more active fractions are then separated again and retested. Inactive fractions are not subject to further purification. This process is iterated until all active antibiotics are isolated. In using this method, the activity of the compounds should be tested using a method which does not result in cleavage of the compounds to release an active antibiotic product of the cleavage, and give a false hit. In other words, steps should be taken to assure that the assay is done on the whole conjugated compound and not on a cleavage product. These precautions are within the skill of the ordinary artisan, and can be determined using routine methods.

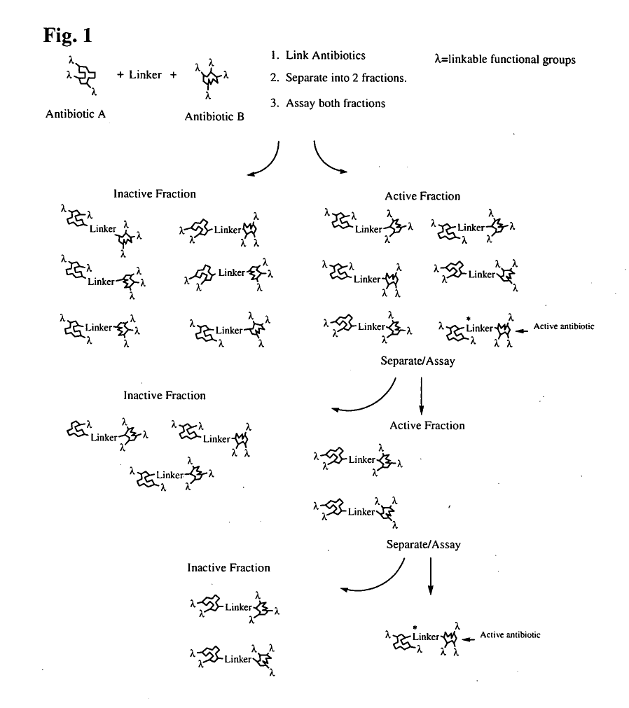

While not intending to limit the scope of the invention in any way, this procedure is demonstrated in a hypothetical example illustrated pictorially in FIG. 1. In FIG. 1, a hypothetical Antibiotic A having 3 linkable functional groups, which are indicated by λ, and a hypothetical Antibiotic B having 4 linkable functional groups, which are also indicated by λ, are linked by a hypothetical linker, indicated in the figure. In this hypothetical example, one of the conjugated molecules is active before biological cleavage, i.e. all three of the intact molecule and the two molecules eventually formed by in vivo cleavage are active antibiotics. The mixture of 12 antibiotics is then separated into two fractions and assayed. The fraction having the active antibiotic is found to be active, and the fraction having no active antibiotic is found to be inactive. The active fraction, consisting of 5 inactive compounds and one active antibiotic in this particular case, is again separated and assayed, to give an active fraction having 3 compounds. Finally, the last separation gives the active antibiotic. Thus, in this hypothetical example, the active compound is identified in three separation/assay steps, which, while not intending to limit the scope of the invention in any way, is likely to be significantly easier than separating the twelve compounds and testing them individually.

Tests for antibiotic activity are well known in the art, and may be chosen according the particular need. For example, U.S. Pat. No. 4,980,470 and U.S. Pat. No. 5,688,792, incorporated herein by reference, give useful methods for making this determination.

Further, disclosed herein are compounds comprising 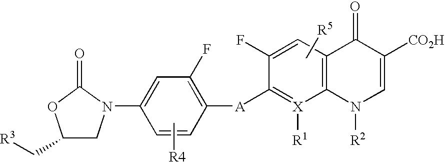
or a pharmaceutically acceptable salt or a prodrug thereof;

wherein A is a linking group comprising an ester or an amide bond X is C or N;

R¹ and R² are independently H, C₁₋₆ alkyl, or C₁₋₆ alkoxy, wherein R¹ and R² may be bonded such that a ring is formed;

R³ is H, C₁₋₆ alkyl, C₁₋₆ acyl, guanidinyl, C₂₋₆ alkylguanidinyl, or C₁₋₆ NH-acyl; and

R⁴ and R⁵ are fluoro, chloro, bromo, nitro, CN, CO₂H, OH, C1-6 alkyl, or C₁₋₆ alkoxy. 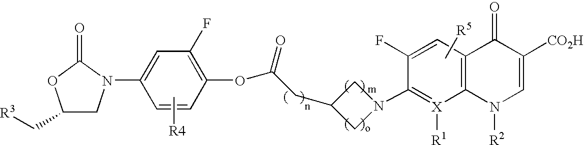
or a pharmaceutically acceptable salt thereof;

wherein

n is from 0 to 3;

and m and o are independently from 0 to 2.

C₁₋₆ alkyl has the meaning normally understood in the art, i.e. hydrocarbon or hydrocarbyl having no double or triple bonds including:

Linear Alkyl such as methyl, ethyl, n-propyl, etc;

Branched Alkyl such as iso-propyl, t-butyl, branched pentyl and hexyl isomers, etc;

Cyclic alkyl such as cyclopropyl, cyclobutyl, etc.; and

Combinations thereof, where any of the above are combined.

C₁₋₆ alkoxy is O—C₁₋₆ alkyl.

C₁₋₆ acyl is 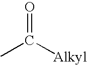
having from 2 to 6 carbon atoms, or formyl.

C₂₋₆ alkylguanidinyl is alkyl having a guanidinyl wherein the alkylguanidinyl has from 2 to 6 carbons, i.e. 1-5 carbon atoms from the alkyl and 1 carbon from the guanidinyl.

C₁₋₆ NH-acyl is C₁₋₆ acyl wherein the carbon atom of the carbonyl moiety is bonded to the nitrogen, and the total number of carbon atoms in the C₁₋₆ NH-acyl is from 1 to 6.

In certain embodiments, R¹ and R² are selected from H, OCH₃, and cyclopropyl.

In other embodiments, R¹ and R² are bonded such that a ring is formed, such as in the compound below.

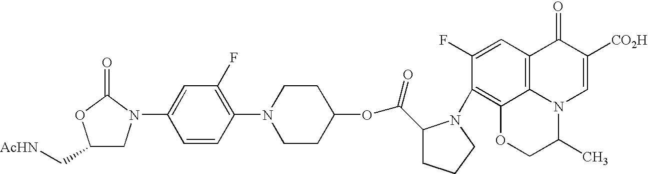

Also contemplated are compounds of a structure shown below, or pharmaceutically acceptable salts or prodrugs thereof. 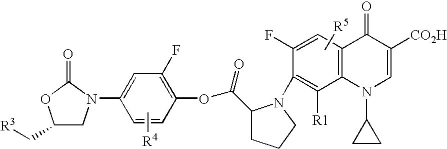
Also contemplated is compound 7 shown below, or a pharmaceutically acceptable salt or a prodrug thereof.

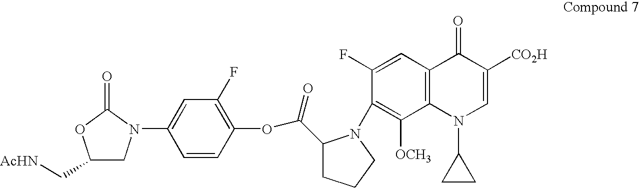

Further, disclosed herein are compounds comprising

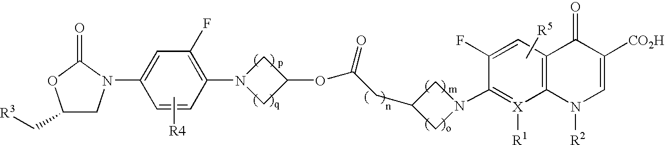

or a pharmaceutically acceptable salt or a prodrug thereof;

wherein n is from 0 to 3;

and m, o, p, and q are independently from 0 to 2.

Also contemplated are compounds of a structure shown below, or pharmaceutically acceptable salts or prodrugs thereof. 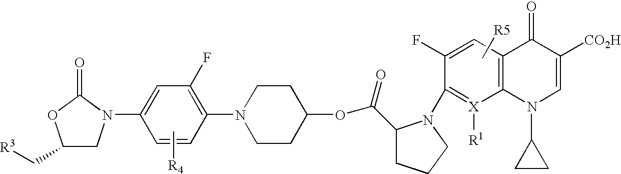
Also contemplated is compound 7 shown below, or a pharmaceutically acceptable salt or a prodrug thereof.

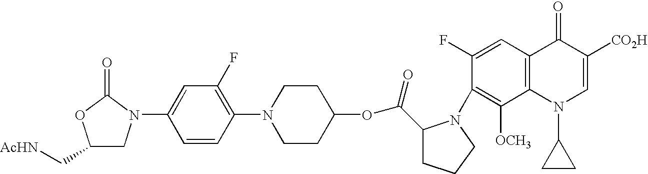

While not intending to be bound in any way by theory, or to limit the scope of the invention in any way, it is believed that these compounds will have oxizolidinone and/or flouroquinolone activity when intact, and will cleave into one or two active antibiotics.

Those skilled in the art will readily understand that for administration or the manufacture of medicaments the compounds disclosed herein can be admixed with pharmaceutically acceptable excipients which per se are well known in the art. Specifically, a drug to be administered systemically, it may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

The amount of the presently useful compound or compounds administered is, of course, dependent on the therapeutic effect or effects desired, on the specific mammal being treated, on the severity and nature of the mammal's condition, on the manner of administration, on the potency and pharmacodynamics of the particular compound or compounds employed, and on the judgment of the prescribing physician.

A liquid composition which is formulated for topical ophthalmic use is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjuster | 1-10 |
| buffer | 0.01-10 |
| pH adjuster | q.s. pH 4.5-7.5 |
| antioxidant | as needed |
| surfactant | as needed |
| purified water | as needed to make 100% |

For topical use, creams, ointments, gels, solutions or suspensions, etc., containing the compound disclosed herein are employed. Topical formulations may generally be comprised of a pharmaceutical carrier, cosolvent, emulsifier, penetration enhancer, preservative system, and emollient.

The actual dose of the active compounds of the present invention depends on the specific compound, and on the condition to be treated; the selection of the appropriate dose is well within the knowledge of the skilled artisan.

Compounds disclosed herein are useful in the treatment of any bacterial infection. Such bacterial infection may affect the ocular tissue, as in conditions including, but not limited to, the following: infectious conjunctivitis, infectious scleritis, ulcerative keratitis, endophthalmitis, and the like.

Other types of bacterial infections that may be treated include bronchitis, pneumonia, sepsis, meningitis, sinusitis, colitis, infectious arthritis infections, and the like.

Any of the compounds disclosed herein may be used in a polymeric implant which is implanted into a body of a mammal. While not intending to limit the scope of the invention in any way, U.S. Pat. No. 5,869,079 describes a suitable type of implant for this purpose. Any type of implant capable of the delivering the compounds disclosed herein is contemplated. In many cases, the implant will be designed to deliver the compound to animal over a sustained period of time by any number of means including diffusion of the compound from the polymer or biodegradation of the polymer. While not intending to be limiting, this type of implant is particularly useful for the targeted delivery of the compound to a particular part of the body for a sustained period of time. While not intending to limit the scope of the invention in any way, this is especially useful where frequent injection of the compound into the particular part of the body is undesirable. For example, frequent injection into or near the eye is undesirable. In particular, frequent injection into the eye is highly undesirable. Thus, an implant comprising the compounds disclosed herein may be placed near or into the eye to deliver the drug over an extended period of time to avoid frequent injections. The term implant should be construed broadly to include devices that are placed on surface where the compound could be absorbed. For example, the implant may also be placed onto the surface of the eye such as in the form of a contact lens. Alternatively, an implant could be placed into the punctum or into the nasolacrimal system, or into any other orifice of a mammal's body.

The following examples illustrate methods of making and using the present invention, and include the best mode contemplated. However, these examples are included purely for illustration, and should not be construed as limiting the scope of the invention in any way.

All starting materials in the following procedures are available commercially.

### EXAMPLE 1

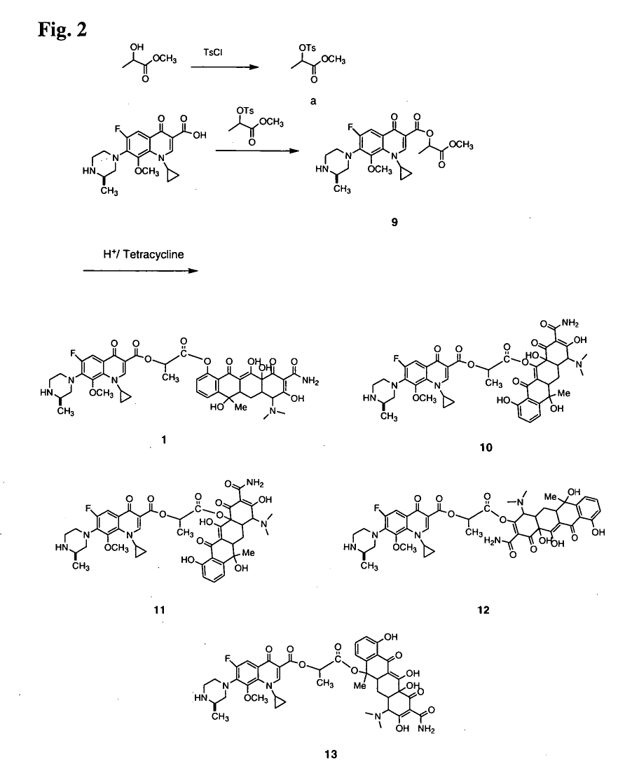

### (FIG. 2)

p-Toluenesulfonyl chloride is stirred with methyl lactate in the presence of pyridine or another suitable base to form compound a, which is then stirred with gatifloxacin to yield a mixture of compounds 1, and 10-13. Compound 1 is isolated by chromatography or some other purification method known in the art.

Alternatively, the mixture of compounds 1, and 10-13 could be subjected to the procedure described previously and depicted in FIG. 1 to isolate a compound which is an active antiobiotic before cleavage occurs in vivo.

### EXAMPLE 2

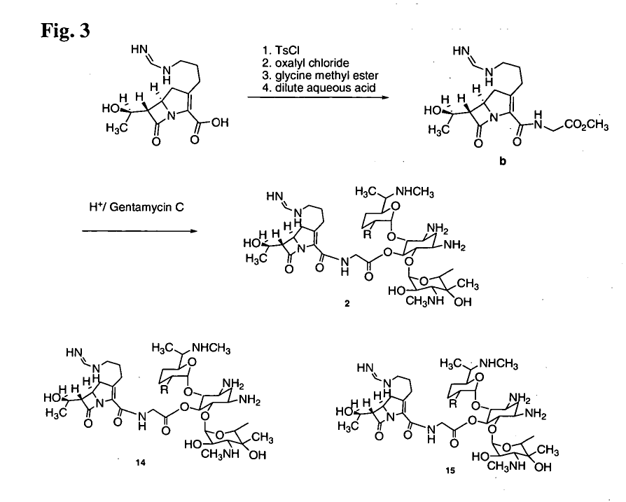

### (FIG. 3)

Imipenem is treated with p-toluensulfonyl chloride, the ipinenem tosylate product is then treated with oxalyl chloride, and the acid chloride is then treated with the methyl ester of glycine followed by dilute aqueous acid to form compound b; Compound b is then subject to transesterification with Gentamycin C to yield a mixture of 2, and 14-15, which is purified by chromatography or some other suitable separation method.

### EXAMPLE 3

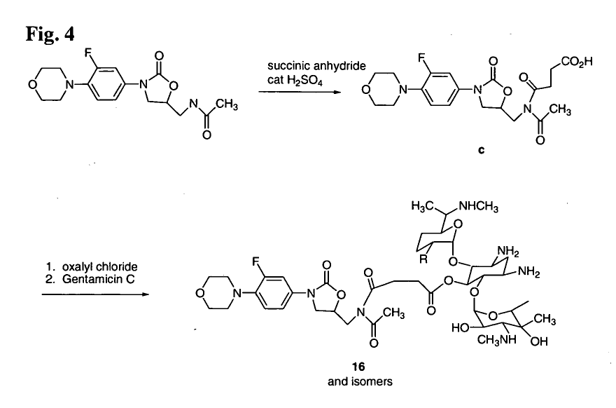

### (FIG. 4)

Linezolid is heated with succinic anhydride in the presence of catalytic sulfuric acid to form compound c. Compound c is then treated with oxalyl chloride and the acid chloride is isolated by distillation or a similar method. Gentamicin C is then added to the acid chloride to form a mixture and compound 16 its isomers. The desired compound is isolated by chromatography or some other suitable method.

### EXAMPLE 4

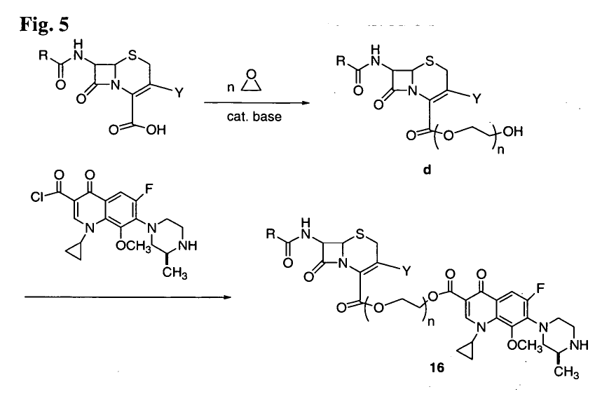

### (FIG. 5)

Cefaclor is treated with an appropriate amount of ethylene oxide in the presence of a catalytic amount of base to produce a statistical mixture of products, from which compound d is isolated by chromatography or some other suitable method. Compound d is treated with the acid chloride (prepared in an analogous manner to the other acid chlorides previously described) to form compound 17, which is purified by chromatography or another suitable method.

### EXAMPLE 5

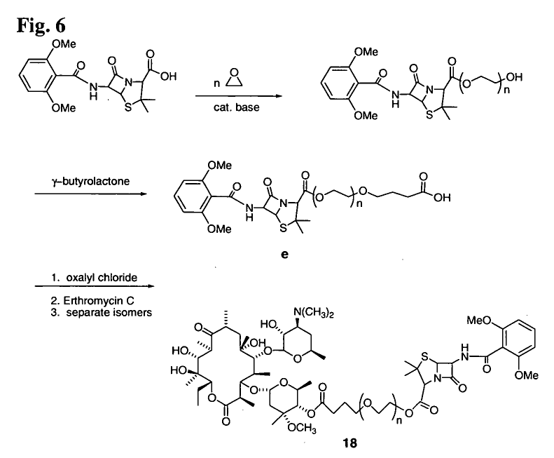

### (FIG. 6)

Methacillin is added to an appropriate amount of ethylene oxide in the presence of a catalytic amount of base, after the reaction is complete, γ-butyrolactone is added to the same pot to form a mixture comprising compound e. Compound e is then treated with oxalyl chloride followed by Erthromycin C to form a mixture of products which include compound 18. Compound 18 is isolated by chromatography or some other suitable method.

### EXAMPLE 6

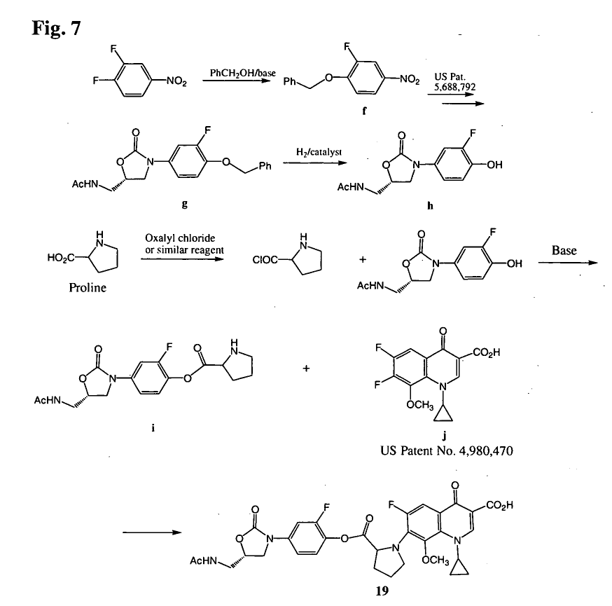

### (FIG. 7)

Benzyl alcohol and a suitable base is added to 3,4-difluoro-1-nitrobenzene, to form compound f which is then worked up and isolated by chromatography or another suitable method. Compound f is then treated according to the procedure of U.S. Pat. No. 5,688,792, incorporated by reference herein, to form compound g. Compound g is deprotected by catalytic hydrogenation to form the phenolic compound h, which is esterified with the acyl chloride of proline to form compound i. Compound i is reacted with compound j, prepared as described in U.S. Pat. No. 4,980,470, incorporated by reference herein, to form compound 19.

### EXAMPLE 7

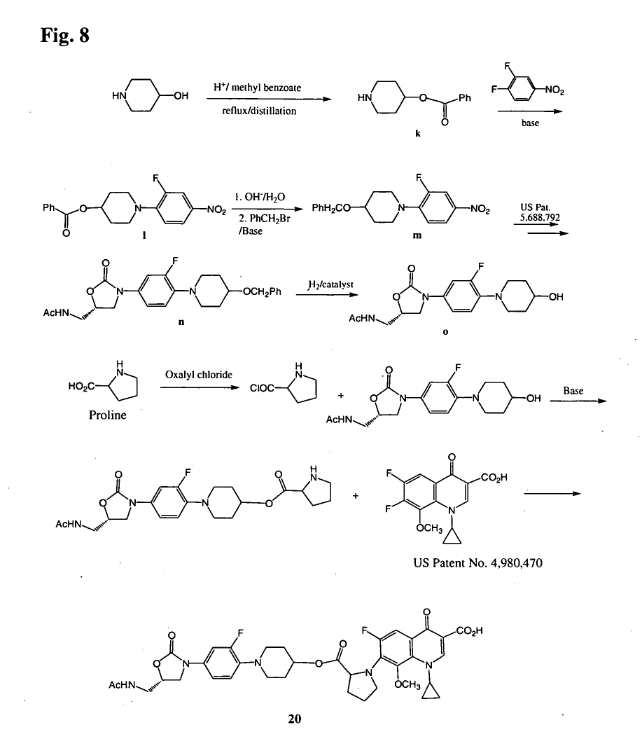

### (FIG. 8)

Methyl benzoate is transesterified with 4-hydroxypiperidine yield compound k. Compound k is then reacted with 3,4-difluoronitrobenzene and a suitable base to give compound 1. Compound 1 is saponified, and then reacted with benzyl bromide under Williamson or equivalent conditions to yield compound m, which is subjected to the procedure of U.S. Pat. No. 5,688,792 to give compound n. The benzylic ether of compound n is deprotected with catalytic hydrogenation to give compound o, which is treated as in the previous example to give compound 20.

### EXAMPLE 8

An eye drop containing compound 1 is administered to a patient suffering from bacterial conjunctivitis over a period of two weeks. After the complete treatment, the bacterial infection is eliminated and relief of symptoms is experienced.

### EXAMPLE 9

An eye drop containing compound 1 is administered to a patient suffering from corneal ulcer over a period of two weeks. After the complete treatment, the bacterial infection is eliminated and relief of symptoms is experienced.

### EXAMPLE 10

An eye drop containing compound 1 is administered to prevent endophthalmitis.

What is claimed is:

1. A compound comprising two antibiotics belonging to distinct classes, which are connected via two covalent bonds to a linker such that said compound degrades in vivo to yield the two antibiotics, wherein each bond is an amide bond or an ester bond.

2. The compound of claim 1 wherein one antibiotic is selected from the group consisting of fluoroquinolones, carbapenems, oxazolidinones, cephalosporin, glycopeptides, and macrolides, and the second antibiotic is selected from the group consisting of tetracyclines, aminoglycosides, fluoroquinolones, and penicillin.

3. The compound of claim 1 wherein said linker comprises an amino acid.

4. The compound of claim 1 wherein said linker comprises lactic acid.

5. The compound of claim 1 wherein said linker comprises ethylene glycol, or an oligomer or polymer thereof.

6. The compound of claim 1 wherein said linker is a polyethylene glycol acid.

7. The compound of claim 1 wherein said antibiotics comprise a fluoroquinone and a tetracycline.

8. The compound of claim 1 wherein said antibiotics comprise a carbapenem and an aminoglycoside.

9. The compound of claim 1 wherein said antibiotics comprise an oxazolidinone and an aminoglycoside.

10. The compound of claim 1 wherein said antibiotics comprise a cephalosporin and a fluoroquinolone.

11. The compound of claim 1 wherein said antibiotics comprise vancomycin and a fluoroquinolone.

12. The compound of claim 1 wherein said antibiotics comprise a macrolide and a penicillin.

13. A composition comprising a compound comprising two antibiotics belonging to distinct classes, which are connected via two covalent bonds to a linker such that said compound degrades in vivo to yield the two antibiotics, wherein each bond is an amide bond or an ester bond, wherein said composition is formulated for topical ophthalmic administration.

14. The composition of claim 13 wherein the pH of said composition is from 4 to 9.

15. A method comprising administration to an eye of a mammal a compound comprising two antibiotics belonging to distinct classes, which are connected via two covalent bonds to a linker such that said compound degrades in vivo to yield the two antibiotics, wherein each bond is an amide bond or an ester bond, wherein said method is effective in the treatment of a bacterial infection affecting said eye.

16. A compound which is an active antibiotic, which degrades in vivo into two or more smaller active antibiotics belonging to distinct classes.

17. The compound of claim 16 wherein said compound has topical antibiotic activity upon a surface of an eye, and wherein the compound degrades on said surface into one or more of said smaller active antibiotics which are capable of penetrating beyond tissue of said surface.

18. A compound comprising a linker having two bonds, wherein said bonds are asymmetrically degraded in vivo to release the two antibiotics belonging to distinct classes.

19. A compound comprising 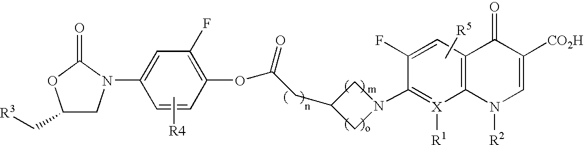
or a pharmaceutically acceptable salt or a prodrug thereof;
wherein R¹ and R² are independently H, C₁₋₆ alkyl, or C₁₋₆ alkoxy, wherein R¹ and R² may be bonded such that a ring is formed;
R³ is H, C₁₋₆ alkyl, C₁₋₆ acyl, guanidinyl, C₂₋₆ alkylguanidinyl, or C₁₋₆ NH-acyl;
R⁴ and R⁵ are fluoro, chloro, bromo, nitro, CN, CO₂H, OH, C₁₋₆ alkyl, or C₁₋₆ alkoxy;
n is from 0 to 3; and
m and o are independently from 0 to 2.

20. The compound of claim 19 comprising 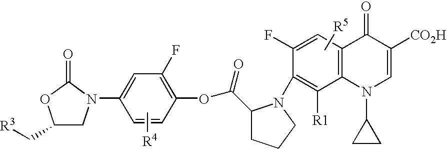
or a pharmaceutically acceptable salt or a prodrug thereof.

21. A compound comprising 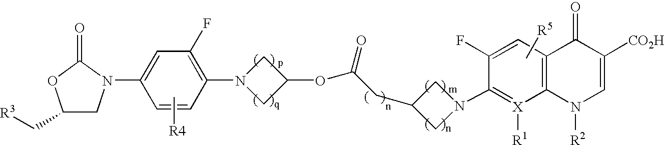
or a pharmaceutically acceptable salt or a prodrug thereof;
wherein R¹ and R² are independently H, C₁₋₆ alkyl, or C₁₋₆ alkoxy, wherein R¹ and R² may be bonded such that a ring is formed;
R³ is H, C₁₋₆ alkyl, C₁₋₆ acyl, guanidinyl, C₂₋₆ alkylguanidinyl, or C₁₋₆ NH-acyl;
R⁴ and R⁵ are fluoro, chloro, bromo, nitro, CN, CO₂H, OH, C₁₋₆ alkyl, or C₁₋₆ alkoxy;
n is from 0 to 3;
and m, o, p, and q are independently from 0 to 2.

22. The compound of claim 21 comprising 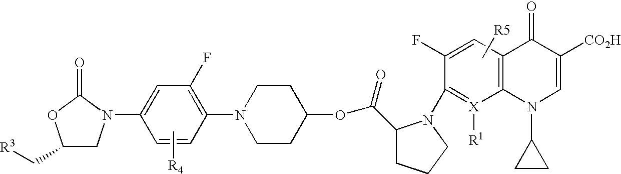
or a pharmaceutically acceptable salt or a prodrug thereof.

23. A method comprising
a. linking two different antibiotics such that a mixture of isomers is formed, wherein one or both antibiotics have more than one linkable group,
b. separating said mixture into two or more fractions,
c. testing the antibiotic activity of said fractions, and
d. repeating steps b and c on the more active fractions;
wherein said method is useful for isolating or identifying a compound which is an active antibiotic.

24. An implant comprising a compound and a polymer wherein said compound degrades into two or more antibiotic compounds in vivo, wherein said polymer provides controlled delivery of said compound for a sustained period of time, and wherein said implant is placed into a body of a mammal.

25. The implant of claim 24 which is implanted into or near an eye.

26. The implant of claim 25 which is implanted into an eye.
